(19) United States
(12) Patent Application Publication
(10) Pub. No.: US 2026/0166015 A1
(43) Pub. Date: Jun. 18, 2026

(54) MODULATION OF NON-MUSCLE MYOSIN IIC (MYH14) FOR REGULATION TEAR AND SALIVA SECRETION

(71) Applicant: University of Virginia Patent Foundation, Charlottesville, VA (US)

(72) Inventors: Seham Ebrahim, Charlottesville, VA (US); Yuta Ohno, Charlottesville, VA (US)

(21) Appl. No.: 19/421,219

(22) Filed: Dec. 16, 2025

Related U.S. Application Data
(60) Provisional application No. 63/735,174, filed on Dec. 17, 2024.

(51) Int. Cl. A61K 31/437; A61K 31/121; A61P 27/02

(52) U.S. Cl. CPC A61K 31/437; A61K 31/121; A61P 27/02

(57) ABSTRACT

The present disclosure describes methods of regulating tear and saliva secretion by modulating non-muscle myosin IIC. In one aspect, disclosed herein is a method of treating a condition associated with tear production in a subject, by administering to the subject a therapeutically effective amount of a non-muscle myosin IIC (NMIIC) modulator, wherein the NMIIC modulator is an NMIIC inhibitor, such as blebbistatin, or pharmaceutically acceptable derivatives. In one aspect, disclosed herein is a method of increasing tear production in a subject, comprising administering to the subject a therapeutically effective amount of an NMIIC inhibitor. In another aspect, disclosed herein is a method treating a condition associated with saliva production in a subject, comprising administering to the subject a therapeutically effective amount of a non-muscle myosin IIC modulator, wherein the NMIIC modulator is an NMIIC activator, such as 4-hydroxyacetophenone, or pharmaceutically acceptable derivatives.

### CROSS-REFERENCE TO RELATED APPLICATIONS

This application claims benefit of priority from U.S. Provisional Application No. 63/735,174, filed on Dec. 17, 2024, the entire contents of which are incorporated herein by reference for all purposes.

### BACKGROUND

### 1. Technical Field

This disclosure provides a method of modulating tear production or saliva production in a subject. The method comprises administering a therapeutically effective amount of a non-muscle myosin (NMIIC) modulator to the subject.

### 2. Brief Description of the Related Art

“Dry eye” is a condition characterized by symptoms of ocular discomfort and visual disturbances due to decreased tear secretion, affecting approximately 16 million Americans. Despite its prevalence, there is currently no cure for dry eye, primarily because the mechanistic details of water secretion in the tear-producing lacrimal gland have not been fully elucidated. While a transcellular water secretion pathway via water channels like AQP5 has been reported, the existence and function of a paracellular pathway via tight junctions between epithelial cells remain controversial.

Tears play a critical role in corneal health by supplying oxygen and nutrients and protecting against bacteria. Reduced tear secretion can lead to “dry eye,” causing blurred vision, burning or itching in the eye, and sensitivity to light. The causes of reduced tear secretion include Sjögren's syndrome, the use of drugs with anticholinergic effects, radiotherapy for head-and-neck tumors, metabolic syndrome, and prolonged exposure to video display terminals. The incomplete understanding of the mechanism of tear secretion has hindered the development of causal treatments for dry eye.

The lacrimal gland, responsible for tear production, mainly comprises two different cell types: acinar cells, which contain secretory granules, and ductal cells that form a tubular network along which secretions are delivered to the surface of the eyeball. There are currently two proposed water secretion pathways: a transcellular water pathway via water channels such as AQP5 and a paracellular water pathway via tight junctions. Evidence from AQP5 deficient mice, which show a half reduction in tear secretion, supports the transcellular pathway but also implies the involvement of other pathways, as tear secretion is not completely abolished. However, the existence and function of the paracellular water pathway remain controversial.

The actomyosin cytoskeleton, which localizes to the apical junctions of epithelial cells across organs, regulates tight junction integrity. Non-muscle myosin IIC (NMIIC) is enriched at the apical junctions of ductal epithelial cells in the lacrimal gland, leading to the hypothesis that NMIIC regulates tear secretion through modulation of tight junction permeability. Consistent with this hypothesis, it was found that tear volume after carbachol stimulation was significantly increased in mice lacking NMIIC, and levels of the tight junction protein ZO-1 were significantly reduced. Furthermore, pharmacological activation of NMIIC by 4-Hydroxyacetophenone in wildtype mice significantly inhibited tear secretion. These findings reveal a paracellular water secretion pathway in the lacrimal gland, regulated by NMIIC-mediated modulation of ductal cell tight junctional permeability, which can be targeted by small molecules.

Therefore, there remains an unmet need for therapeutic strategies that modulate tear secretion by directly regulating paracellular water transport in the lacrimal gland. In particular, there is a need for methods and compositions that target NMIIC-mediated control of tight junction permeability in ductal epithelial cells, thereby enabling restoration or enhancement of tear production.

### SUMMARY

Disclosed herein is a method of treating a condition associated with tear production in a subject. The method comprises administering to the subject a therapeutically effective amount of a non-muscle myosin IIC (NMIIC) modulator, wherein the NMIIC modulator is an NMIIC inhibitor. The NMIIC inhibitor comprises Blebbistatin (CAS Reg. No. 856925-71-8), MT-228 (CAS Reg. No. 2404652-24-8), or a pharmaceutically acceptable salt, solvate, or hydrate thereof of any of the foregoing.

In one aspect, disclosed herein is a method of increasing tear production in a subject. The method comprises administering to the subject a therapeutically effective amount of a non-muscle myosin IIC (NMIIC) modulator, wherein the NMIIC modulator is an NMIIC inhibitor. The NMIIC inhibitor comprises Blebbistatin (CAS Reg. No. 856925-71-8), MT-228 (CAS Reg. No. 2404652-24-8), or a pharmaceutically acceptable salt, solvate, or hydrate thereof of any of the foregoing.

In another aspect, disclosed herein is a method treating a condition associated with saliva production in a subject. The method comprises administering to the subject a therapeutically effective amount of a non-muscle myosin IIC modulator, wherein the NMIIC modulator is an NMIIC activator. The NMIIC activator comprises 4-hydroxyacetophenone (CAS Reg. No. 99-93-4), or a pharmaceutically acceptable salt, solvate, or hydrate thereof.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1: NMIIC is enriched at tight junctions between ductal cells in murine lacrimal gland. Myosin heavy chain (Myh) mRNA expression level in the murine lacrimal gland from previous RNA-seq data (accession number DRA010121 at DDBJ Sequenced Read Archive).

FIG. 2A: Illustration of procedure for tear volume measurement. The lacrimal gland of anesthetized mouse was surgically exposed wrapped with a KIM wipe upon which carbachol (CCh) was applied. A phenol red-impregnated thread was placed at the canthus of eye for 3 min each before and after CCh stimulation, and tear volume was measured as the length of the thread that turned red due to exposure to moisture. Created with BioRender.com. FIG. 2B: Tear volume from NMIIC-KO mice and WT mice. FIG. 2C: Incremental tear volume from NMIIC-KO mice and WT mice, calculated by subtracting tear volume before CCh stimulation from tear volume after CCh stimulation. **P<0.01. Two-way ANOVA followed by Sidak's multiple comparison test or t-test. N=10-12. FIG. 2D: Western blot of NMIIC, AQP5, Zonula Occludens-1 (ZO-1), and α-tubulin in lacrimal gland from NMIIC-KO mice and WT mice, after CCh stimulation to the lacrimal gland or without CCh. FIG. 2E: Quantification of AQP5 band intensity. FIG. 2F: Quantification of ZO-1 band intensity. **P<0.05, **P<0.01, ***P<0.001, ****P<0.0001. Two-way ANOVA followed by Fisher's LSD test. N=3 or 4.

FIG. 3A: Illustration of collection of tears after lacrimal gland stimulation with CCh-fluorescein mixture. Tears were collected and put on the nitrocellulose membrane and then observed under the microscope. Created with BioRender.com. FIG. 3B: Quantification of relative fluorescence intensity in the same field of view of microscope. *P<0.05. Unpaired t-test with Welch's correction. N=4. FIG. 3D: Protocol of 4-HAP treatment. 4-HAP was administered at 1 mg/kg, i.p., to WT mice once a day for 7 days before measuring tear volume. FIG. 3E: Tear volume from 4-HAP-treated mice and vehicle-treated mice. FIG. 3F: Incremental tear volume from 4-HAP-treated mice and vehicle-treated mice, calculated by subtracting tear volume before CCh stimulation from tear volume after CCh stimulation. *P<0.05, **P<0.01. Two-way ANOVA followed by Sidak's multiple comparison test or t-test. N=7 or 8.

FIG. 4A: Comparison of NMIIC expression level between lacrimal glands and parotid glands by western blot. FIG. 4B: Quantification of NMIIC band intensity. **P<0.01. Student's t-test. N=4 or 5. FIG. 4C: Illustration of procedure for saliva volume measurement. The parotid gland of anesthetized mouse was surgically exposed wrapped with a KIM wipe upon which CCh was applied. Rolled paper was inserted into oral cavity before and after CCh stimulation every 3 min up to 15 min, and saliva volume was measured as the weight of absorbed saliva. Created with BioRender.com. FIG. 4D: Time-dependent change of saliva volume from NMIIC-KO mice and WT mice. FIG. 4E: Total saliva volume for 15 min after CCh stimulation from NMIIC-KO mice and WT mice. *P<0.05, **P<0.01. Two-way ANOVA followed by Sidak's multiple comparison test (FIG. 4D) or t-test (FIG. 4E). N=4. FIG. 4F: Western blot of NMIIC, AQP5, ZO-1, and α-tubulin in parotid gland from NMIIC-KO mice and WT mice without CCh stimulation. FIG. 4G: Quantification of AQP5 band intensity. FIG. 4H: Quantification of ZO-1 band intensity. *P<0.05. Student's t-test. N=3-5.

FIG. 5: Model for NMIIC-mediated regulation of paracellular water transport in tear and saliva secretion in mice NMIIC is enriched at ductal cell tight junctions in the murine lacrimal and salivary glands (parotid glands). Loss of NMIIC results in reduced expression of ZO-1 in both glands. In the lacrimal gland, water flows through AQP5 channels and tight junctions towards the lumen due to higher ion concentration and positive osmotic pressure. Ablation of NMIIC, and consequential reduction of ZO-1 and loosening of tight junctions causes increased water flow and thus tear secretion increases. Conversely, in the salivary gland ductal cells do not express water channels such as AQP5. Thus, while ions are pumped back into ductal cells from the lumen, water remains, resulting in a negative osmotic pressure in the lumen. In this case when tight junctional permeability is increased in absence of NMIIC, water flows out of the lumen resulting in reduced saliva secretion. Created with BioRender.com.

FIG. 6A: Western blot of NMIIC, AQP5, ZO-1, and α-tubulin in lacrimal gland from 4-HAP- and vehicle-injected mice without CCh stimulation. 4-HAP-injected mice showed no NMIIC protein band and similar expression level of AQP5 and ZO-1 to vehicle-injected mice.

FIG. 6B: Quantification of AQP5 band intensity. FIG. 6C: Quantification of ZO-1 band intensity. Student's t-test. N=4.

### DETAILED DESCRIPTION OF THE INVENTION

### General Disclosure

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

The terms “a” and “an” do not denote a limitation of quantity, but rather denote the presence of at least one of the referenced item. The term “or” means “and/or”. The open-ended transitional phrase “comprising” encompasses the intermediate transitional phrase “consisting essentially of” and the close-ended phrase “consisting of” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Where a combination is disclosed, it is understood that each possible sub-combination of the elements of that combination is also disclosed. Conversely, where different elements or groups of elements are individually disclosed, combinations thereof are also disclosed.

Where elements are presented in list format or as alternative members of a group (e.g., a Markush group), it is understood that each possible subgroup of the elements is also disclosed, and any one or more elements can be removed from the list or group.

Where a range of numerical values is recited, it is understood that the endpoints and each intervening integer value and each fraction thereof, as well as each subrange, between the recited endpoints (upper and lower limits) of that range are specifically disclosed. The endpoints of all ranges are included within the range and are independently combinable. Where a value has an inherent limit, that inherent limit is specifically disclosed. Where a value is explicitly recited, it is understood that values which are about the same as the recited value are specifically disclosed.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether the specific exclusion is recited in the specification.

In addition, it is understood that any functional language used in any claims shall not be construed as “means-plus-function” language under 35 U.S.C. § 112(f), unless specifically expressed as such by use of the term “means for” or “step(s) for” in a claim.

It is further understood that the present disclosure encompasses analogs, derivatives, prodrugs, metabolites, salts, solvates, hydrates, clathrates and polymorphs of all the compounds/substances disclosed herein, as appropriate. The specific recitation of “analogs”, “derivatives”, “prodrugs”, “metabolites”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned or shown without recitation of any of these forms, unless stated otherwise or the context clearly indicates otherwise.

It is also understood that the present disclosure encompasses all possible tautomers, all possible regioisomers, and all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds/substances described herein as appropriate, and not only the specific tautomers, regioisomers and stereoisomers as indicated by drawn structure or nomenclature. Some embodiments of the disclosure relate to the specific tautomers, regioisomers and stereoisomers indicated by drawn structure or nomenclature. The specific recitation of the phrase “or tautomers thereof”, “or regioisomers thereof”, “or stereoisomers thereof” or the like with respect to a compound/substance or a group of compounds/substances in certain instances of the disclosure shall not be interpreted as an intended omission of any of the other possible tautomers, regioisomers and stereoisomers of the compound/substance or the group of compounds/substances in other instances of the disclosure where the term “compound” or the like is used, or where the compound/substance or the group of compounds/substances is mentioned or shown, without recitation of the phrase “or tautomers thereof”, “or regioisomers thereof”, “or stereoisomers thereof” or the like, unless stated otherwise or the context clearly indicates otherwise.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

The terms “or/and” and “and/or” mean “either . . . or . . . , or both . . . and . . . ” when referring to two elements, and mean “either . . . , . . . or . . . , or any combination or all thereof” when referring to three or more elements. As an example, the phrase “A or/and B” means “either A or B, or both A and B”, and the phrase “A, B or/and C” means “either A, B or C, or any combination or all thereof”.

As used in the specification and the claims, all transitional terms such as “comprising”, “containing”, “having”, “including”, “possessing”, “holding”, “carrying”, “bearing”, “composed of”, “characterized by” and the like are open-ended and inclusive, that is, mean including but not limited to and do not exclude additional, unrecited element(s) or method step(s). Only the transitional term “consisting of” is closed, that is, excludes any additional, unrecited element or method step, and the transitional term “consisting essentially of” is semi-closed, that is, only allows inclusion of additional, unrecited element(s) or method step(s) that do not materially affect the basic and novel characteristic(s) of that particular embodiment.

The term “exemplary” as used herein means “serving as an example, instance or illustration”. Any embodiment or feature characterized herein as “exemplary” should not be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “pharmaceutically acceptable” means that a substance (e.g., an active ingredient or an excipient) is generally safe, non-toxic and suitable for use in contact with the cells, tissues and organs of a subject without excessive irritation, allergic response, immunogenicity and other adverse reaction. A “pharmaceutically acceptable” excipient or carrier of a pharmaceutical composition is also compatible with the other ingredients of the composition.

The term “therapeutically effective amount” refers to an amount of a compound that, when administered to a subject or used ex vivo, is sufficient to prevent, reduce the risk of developing, delay the onset of, slow the progression of or cause regression of the medical condition being treated, or to alleviate or ameliorate to some extent the medical condition or one or more symptoms or complications of that condition, at least in some fraction of the subjects taking that compound or undergoing ex vivo treatment with that compound. The term “therapeutically effective amount” also refers to an amount of a compound that is sufficient to elicit the biological or medical response of a cell, tissue, organ, system, animal or human which is sought by a researcher, veterinarian, medical doctor or clinician.

The terms “treat”, “treating” and “treatment” include alleviating, ameliorating, reducing the incidence, frequency or severity of, slowing or stopping the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk or likelihood of developing, and delaying the onset of a medical condition or one or more symptoms or complications associated with the condition.

The term “medical conditions” (or “conditions” for brevity) includes diseases and disorders. The terms “diseases” and “disorders” are used interchangeably herein.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a bovine (e.g., a cattle), a suid (e.g., a pig), a caprine (e.g., a sheep), an equine (e.g., a horse), a canine (e.g., a dog) or a feline (e.g., a cat). The terms “subject” and “patient” may be used interchangeably herein in reference to a subject/patient (e.g., a mammalian subject/patient such as a human subject/patient) having a medical condition.

The term “compound” or the like (e.g., “molecule”) encompasses salts, solvates, hydrates, polymorphs, and isotopically labelled forms of that compound. A “solvate” of a compound comprises a stoichiometric or non-stoichiometric amount of a solvent molecule (e.g., water, acetone or an alcohol [e.g., ethanol]) bound non-covalently to the compound. A “hydrate” of a compound comprises a stoichiometric or non-stoichiometric amount of water molecule bound non-covalently to the compound. A “polymorph” of a compound is a crystalline form of the compound. The specific recitation of “salt”, “solvate”, “hydrate”, or “polymorph” with respect to a compound or a group of compounds in certain instances of the disclosure shall not be interpreted as an intended omission of any of these forms in other instances of the disclosure where the term “compound” or the like (e.g., “molecule”) is used, or where the compound or the group of compounds is mentioned or shown, without recitation of any of these forms, unless stated otherwise or the context clearly indicates otherwise.

### Pharmaceutical Compositions

“Pharmaceutical compositions” are compositions comprising at least one active agent, such as a compound or salt of a non-muscle myosin IIC (NMIIC) modulator, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs.

In some embodiments, the NMIIC modulator is an NMIIC inhibitor. The NMIIC inhibitor comprises blebbistatin (CAS Reg. No. 856925-71-8 or 674289-55-5 for the racemic mixture), MT-228 (CAS Reg. No. 2404652-24-8), or a pharmaceutically acceptable salt, solvate, or hydrate thereof of any of the foregoing. Blebbistatin was first disclosed by A. Straight, et al., (Science. 2003; 299:1743-1747), MT-228 was disclosed in US20210317117 (U.S. Pat. No. 11,649,234), which is hereby incorporated by reference for its disclosure of NMIIC inhibitors. Blebbistatin analogues are disclosed in US 20210087201 (U.S. Ser. No. 11/746,112) which is hereby incorporated by reference for its disclosure of NMII inhibitors, including NMIIC inhibitors.

In some embodiments, the NMIIC modulator is an NMIIC activator. The NMIIC activator comprises 4-hydroxyacetophenone (CAS Reg. No. 99-93-4), or a pharmaceutically acceptable salt, solvate, or hydrate thereof.

“Pharmaceutically acceptable salts” includes derivatives of the disclosed non-muscle myosin IIC (NMIIC) modulator in which the parent compound is modified by making inorganic and organic, non-toxic, acid or base addition salts thereof. The salts of the non-muscle myosin IIC modulator can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

Where a compound exists in various tautomeric forms, the invention is not limited to any one of the specific tautomers, but rather includes all tautomeric forms.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any excipient or carrier is incompatible with the active ingredient, the disclosure encompasses the use of conventional excipients and carriers in formulations containing the non-muscle myosin IIC modulators. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

### Methods of Non-Muscle Myosin Iic Modulators Administration

In certain embodiments, the NMIIC modulator is administered topically to an ocular surface of the subject. The modulator may be formulated for topical ophthalmic delivery in a suitable dosage form, including an ophthalmic solution, suspension, gel, ointment, or emulsion. These formulations may be adapted for immediate or sustained release, and may contain pharmaceutically acceptable excipients suitable for ocular administration.

The NMIIC modulator may be provided in a variety of ophthalmic delivery systems, such as a unit-dose sterile ampoule, a multi-dose preservative-free eye drop container, or an ophthalmic squeeze dispenser configured to deliver accurate drop volumes to the eye. Dosing regimens may vary depending on the severity of the condition, and in some embodiments the modulator is administered once daily, twice daily, or three times daily.

The concentration of the NMIIC modulator in the ophthalmic formulation may range from 0.0001% to 5% weight/volume (w/v). Systemic exposure or topical delivery may be adjusted to achieve a therapeutically effective dose, which in some embodiments corresponds to 0.001 mg/kg to 50 mg/kg of the NMIIC modulator.

A therapeutically effective amount of the NMIIC modulator may be defined as an amount sufficient to increase tear volume, increase aqueous tear secretion, or improve tear film stability relative to a pre-treatment baseline. These therapeutic improvements may be measurable by clinical tests such as Schirmer's test, tear-film breakup time, or imaging-based tear-film assessment.

In some embodiments, the NMIIC modulator is delivered using a controlled-release topical ocular dosage form, including but not limited to a gel-forming in situ formulation, an ocular insert, a hydrogel depot, or a biodegradable carrier configured to release the modulator over an extended period.

The NMIIC modulator may also be co-administered with one or more additional therapeutic agents to enhance lubrication, reduce inflammation, or modulate ocular surface immunity. Exemplary co-therapies include a lubricant, a corticosteroid, an immunomodulator, or an anti-inflammatory agent, administered concurrently or sequentially with the NMIIC inhibitor.

In some embodiments, the NMIIC modulator is formulated for topical delivery within the oral cavity. The activator may be provided in a mucosal gel, an oral rinse, a spray, a film, a paste, or a bioadhesive formulation, each of which is suitable for contacting oral mucosal surfaces and delivering the active agent locally to tissues involved in saliva production. Such formulations may include mucoadhesive polymers, wetting agents, or penetration enhancers to promote residence time and effective absorption at the administration site.

Appropriate formulation can depend on various factors, such as the route of administration chosen. Potential routes of administration of pharmaceutical compositions include without limitation oral, parenteral (including intradermal, subcutaneous, intramuscular, intravascular, intravenous, intraarterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect. In some embodiments, the non-muscle myosin IIC modulators are administered as an oral dosage form such as a tablet, capsule or pill.

As an example, formulations of the non-muscle myosin IIC modulators suitable for oral administration can be presented in discrete units adapted for instant, controlled or sustained release as, e.g., boluses; capsules (including push-fit capsules and soft capsules), tablets, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Push-fit capsules or two-piece hard gelatin capsules can contain non-muscle myosin IIC modulators in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, non-muscle myosin IIC modulators can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of non-muscle myosin IIC modulators can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

The non-muscle myosin IIC modulators can also be formulated for parenteral administration by injection or infusion to circumvent gastrointestinal absorption and first-pass metabolism. An exemplary parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain non-muscle myosin IIC modulators along with excipients such as an antioxidant, a buffer, a bacteriostat and solutes that render the formulation isotonic with the blood of the subject. Aqueous or non-aqueous sterile suspensions can contain non-muscle myosin IIC modulators along with excipients such as a suspending agent and a thickening agent, and optionally a stabilizer and an agent that increases the solubility of the non-muscle myosin IIC modulators to allow for the preparation of a more concentrated solution or suspension. As another example, a sterile aqueous solution for injection or infusion (e.g., subcutaneously or intravenously) can contain non-muscle myosin IIC modulators, an isotonic agent (e.g., sodium chloride), a buffering agent (e.g., sodium citrate), a preservative (e.g., meta-cresol), and optionally a base (e.g., NaOH) or/and an acid (e.g., HCl) to adjust pH.

Topical formulations for application to the skin or mucosa can be useful for transdermal or transmucosal administration of a therapeutic agent to the local target site of action, or into the blood for systemic distribution. Advantages of topical administration can include circumvention of the GI tract (including enzymes and acid in the GI tract and absorption through it) and first-pass metabolism; delivery of a therapeutic agent with a short half-life, a small therapeutic index or/and low oral bioavailability; controlled, continuous and sustained release of the therapeutic agent; a more uniform plasma level or delivery profile of the therapeutic agent; lower dose and less frequent dosing of the therapeutic agent; reduction of systemic side effects (e.g., side effects caused by a temporary overdose or an overly high peak plasma drug concentration); minimal or no invasiveness; ease of self-administration; and increased patient compliance.

Compositions suitable for topical administration include without limitation liquid or semi-liquid preparations such as sprays, gels, liniments, lotions, oil-in-water or water-in-oil emulsions such as creams, foams, ointments and pastes, and solutions or suspensions such as drops (e.g., eye drops, nose drops and ear drops). See Remington: The Science and Practice of Pharmacy, 21st Ed., Lippincott Williams & Wilkins (Philadelphia, Pennsylvania [2005]). Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g. menthol), opacifiers, fragrances and colorants. For an active agent having a low rate of permeation through the skin or mucosal tissue, a topical formulation can contain a chemical permeation enhancer (e.g., a fatty acid ester [e.g., isopropyl myristate or isopropyl palmitate], a fatty acid [e.g., palmitic acid, oleic acid or palmitoleic acid], or/and an alcohol [e.g., propylene glycol or a fatty alcohol such as geraniol or farnesol]) to increase the permeation of the active agent through the skin or mucosal tissue. A topical formulation can also contain an irritation-mitigating excipient that reduces any irritation to the skin or mucosa caused by the active agent, the chemical permeation enhancer or any other component of the formulation. In some embodiments, a topical composition comprises a therapeutic agent dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]), a gel, an emulsifier, a thickening agent, a stabilizer or a preservative, or any combination thereof. A topical formulation can be administered by means of, e.g., a transdermal or transmucosal delivery device, such as a transdermal patch, a microneedle patch or an iontophoresis device. A topical composition can deliver a drug transdermally or transmucosally via a concentration gradient (with or without the use of a chemical permeation enhancer) or an active mechanism (e.g., iontophoresis or microneedles).

For topical administration, the non-muscle myosin IIC modulators can be formulated as, e.g., a buccal or sublingual tablet or pill. Advantages of a buccal or sublingual tablet or pill include avoidance of gastrointestinal absorption and first-pass metabolism, and rapid absorption into systemic circulation. A buccal or sublingual tablet or pill can be designed to provide faster release of the non-muscle myosin IIC modulators for more rapid uptake of it into systemic circulation. In addition to a therapeutically effective amount of non-muscle myosin IIC modulators, the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

In some embodiments, the non-muscle myosin IIC modulators are administered transdermally. In some embodiments, the topical composition or the transdermal delivery system comprises a chemical permeation enhancer (e.g., a surfactant [e.g., sodium laureth sulfate], optionally in combination with an aromatic compound [e.g., phenylpiperazine]) that facilitates the transport of the non-muscle myosin IIC modulators across the skin. In further embodiments, the non-muscle myosin IIC modulators are administered via a transdermal patch. In some embodiments, the transdermal patch is a reservoir-type patch comprising an impermeable backing layer/film, a liquid- or gel-based drug reservoir, a semi-permeable membrane that serves as a rate-limiting or rate-controlling diffusion barrier, and a skin-contacting adhesive layer. The semi-permeable membrane can be composed of, e.g., a suitable polymeric material such as cellulose nitrate or acetate, polyisobutene, polypropylene, polyvinyl acetate or a polycarbonate. In other embodiments, the transdermal patch is a drug-in-adhesive patch comprising an impermeable backing layer/film and a skin-contacting adhesive layer incorporating the drug in a polymeric or viscous adhesive. The adhesive of the drug-loaded, skin-contacting adhesive layer can be, e.g., a pressure-sensitive adhesive (PSA), such as a PSA composed of an acrylic polymer (e.g., polyacrylate), a polyalkylene (e.g., polyisobutylene) or a silicone-based polymer (e.g., silicone-2675 or silicone-2920). Transdermal drug-delivery systems, including patches, can be designed to provide controlled and prolonged release of a drug over a period of about 1 week, 2 weeks, 3 weeks, 1 month or longer.

In some embodiments, the non-muscle myosin IIC modulators are delivered from a sustained-release composition. As used herein, the term “sustained-release composition” encompasses sustained-release, prolonged-release, extended-release, delayed-release, slow-release and controlled-release compositions, systems and devices. Advantages of a sustained-release composition include without limitation a more uniform blood level of the drug (e.g., avoidance of wide peak-to-trough fluctuations), delivery of a therapeutically effective amount of the drug over a prolonged time period, reduced frequency of administration, and reduced side effects (e.g., avoidance of a drug overdose). In some embodiments, the sustained-release composition delivers the non-muscle myosin IIC modulators over a period of at least about 1 day, 2 days, 3 days, 1 week, 2 weeks, 3 weeks, 1 month, 2 months, 3 months or longer.

In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is formulated as polymeric nanoparticles or microparticles, wherein the polymeric particles can be delivered, e.g., by injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

In further embodiments, the sustained-release composition comprises a dendrimer. In certain embodiments, the dendrimer is a water-soluble dendrimer, such as a poly(amidoamine) (PAMAM) dendrimer. In some embodiments, a dendrimer encapsulates a drug through the formation of a dendrimer-drug supramolecular assembly. In other embodiments, the sustained-release composition comprises a water-soluble polymer [e.g., poly(DL-lactide)] or a liposome encapsulating a drug complexed with a dendrimer.

In other embodiments, the sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

For a delayed or sustained release of non-muscle myosin IIC modulators, a non-muscle myosin IIC modulator can also be formulated as, e.g., a depot that can be implanted in or injected into a subject, e.g., intramuscularly, intracutaneously or subcutaneously. A depot formulation can be designed to deliver non-muscle myosin IIC modulators over an extended period of time, e.g., over a period of at least about 1 week, 2 weeks, 3 weeks, 1 month, 6 weeks, 2 months, 3 months or longer. For example, non-muscle myosin IIC modulators can be formulated with a polymeric material (e.g., polyethylene glycol [PEG], polylactic acid [PLA] or polyglycolic acid [PGA], or a copolymer thereof [e.g., PLGA or PLA-PEG]), with a hydrophobic material (e.g., as an emulsion in an oil) and/or an ion-exchange resin, as a more lipophilic derivative (e.g., as an ester of or a salt with a fatty acid such as a C₈-C₂₀ fatty acid [e.g., decanoic acid]), or as a sparingly soluble derivative (e.g., a sparingly soluble salt). As an illustrative example, non-muscle myosin IIC modulators can be incorporated or embedded in sustained release microparticles composed of PLGA and formulated as a monthly depot.

The non-muscle myosin IIC modulators can also be contained or dispersed in a matrix material. The matrix material can comprise a polymer (e.g., ethylene-vinyl acetate) and controls the release of the drug by controlling dissolution and/or diffusion of the drug from, e.g., a reservoir, and can enhance the stability of the drug while contained in the reservoir. Such a release system can be designed as a sustained-release system, can be configured as, e.g., a transdermal or transmucosal patch, and can contain an excipient that can accelerate the drug's release, such as a water-swellable material (e.g., a hydrogel) that aids in expelling the drug out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a drug through it over an extended time period. In addition, continuous release can be approximated by releasing several pulses of a drug in rapid succession (“digital” release). An active release system can be used alone or in conjunction with a passive release system, as described in U.S. Pat. No. 5,797,898.

In addition, pharmaceutical compositions comprising non-muscle myosin IIC modulators can be formulated as, e.g., liposomes, micelles (e.g., those composed of biodegradable natural or/and synthetic polymers, such as lactosomes), nanoparticles (e.g., lipid nanoparticles such as solid lipid nanoparticles), microparticles or microspheres, whether or not designed for sustained release. For example, liposomes can be used as sustained-release pulmonary drug-delivery systems that deliver drugs to the alveolar surface for treatment of lung diseases and systemic diseases. As another example, lipid nanoparticles containing a lipophilic drug can be delivered into the lungs by oral inhalation for treatment of a lung disorder or a systemic disorder.

In some embodiments, liposomes or micelles are composed of one or more phospholipids. Phospholipids include without limitation phosphatidic acids (e.g., DEPA, DLPA, DMPA, DOPA, DPPA and DSPA), phosphatidylcholines (e.g., DDPC, DEPC, DLPC, DLOPC, DMPC, DOPC, DPPC, DSPC, MPPC, MSPC, PLPC, PMPC, POPC, PSPC, SMPC, SOPC and SPPC), phosphatidylethanolamines (e.g., DEPE, DLPE, DMPE, DOPE, DPPE, DSPE and POPE), phosphatidylglycerols (e.g., DEPG, DLPG, DMPG, DOPG, DPPG, DSPG and POPG), phosphatidylserines (e.g., DLPS, DMPS, DOPS, DPPS and DSPS), and salts (e.g., sodium and ammonium salts) thereof. In certain embodiments, liposomes or micelles are composed of one or more phosphatidylcholines. Liposomes have a hydrophilic core, so liposomes are particularly suited for delivery of more hydrophilic drugs, whereas micelles have a hydrophobic core, so micelles are particularly suited for delivery of more hydrophobic drugs. Liposomes and micelles can permeate across biological membranes. Moreover, liposomes and micelles composed of a fusogenic lipid (e.g., DPPG) can fuse with the plasma membrane of cells and thereby deliver a drug into those cells. Liposomes and micelles can provide sustained release of a drug based in part on the rate of degradation of the liposomes and micelles.

The non-muscle myosin IIC modulators can be presented in unit dosage form as a single dose wherein all active and inactive ingredients are combined in a suitable system, and components do not need to be mixed to form the non-muscle myosin IIC modulators to be administered. A unit dosage form generally contains a therapeutically effective dose of the drug, but can contain an appropriate fraction thereof so that taking multiple unit dosage forms achieves the therapeutically effective dose. Examples of a unit dosage form include a tablet, capsule, or pill for oral uptake; a solution in a pre-filled syringe of a single-use pen or a pen with a dose counter for parenteral (e.g., intravenous, subcutaneous or intramuscular) injection; a capsule, cartridge or blister pre-loaded in or manually loaded into an inhaler; and a reservoir-type transdermal patch or a drug-in-adhesive patch.

Alternatively, the non-muscle myosin IIC modulators can be presented as a kit in which the active ingredient, excipient(s) and carrier(s) [e.g., solvent(s)] are provided in two or more separate containers (e.g., ampules, vials, tubes, bottles or syringes) and need to be combined to form the composition to be administered. The kit can contain instructions for storing, preparing and administering the composition (e.g., a solution to be injected parenterally).

A kit can contain all active and inactive ingredients in unit dosage form or the active ingredient and inactive ingredients in two or more separate containers, and can contain instructions for administering or using the pharmaceutical composition to treat a medical condition. A kit can further contain a device for delivering the composition, such as a needle and a syringe, an injection pen, an inhaler or a transdermal patch.

In some embodiments, a kit contains non-muscle myosin IIC modulators or a pharmaceutically acceptable salt, solvate, hydrate, clathrate or polymorph thereof, or a pharmaceutical composition comprising the same, and instructions for administering or using the non-muscle myosin IIC modulators or the composition to treat a medical condition (e.g., a tumor or cancer, a metabolic disorder or a liver disorder). In some embodiments, the kit further contains a device for delivering the non-muscle myosin IIC modulators or the composition, such as an injection pen, an inhaler or a transdermal patch.

Another aspect of the invention relates to a pharmaceutical composition that includes the non-muscle myosin IIC modulators, as described herein, in an aqueous medium.

In certain embodiments, the non-muscle myosin IIC modulators in the pharmaceutical composition form micelle structures. Additionally, the pharmaceutical composition can further contain a therapeutic agent encapsulated within the micelle structures. These therapeutic agents can be hydrophobic.

### Therapeutic Uses of Non-Muscle Myosin IIC Modulators

This disclosure includes methods of treating conditions in which a patient suffers from dry eyes, or dry eye syndrome, inadequate tear productions. Medical conditions associated with Dry Eyes comprise:

Sjögren's Syndrome. An autoimmune disorder characterized by dry eyes and dry mouth due to the body's immune system attacking its own moisture-producing glands.

Use of Drugs with Anticholinergic Effects. Medications such as antihistamines, antidepressants, and certain blood pressure medications can reduce tear production and lead to dry eyes.

Radiotherapy for Head-and-Neck Tumors. Radiation treatment in the head and neck region can damage the lacrimal glands, leading to decreased tear production and dry eyes.

Metabolic Syndrome. A cluster of conditions including high blood pressure, high blood sugar, excess body fat around the waist, and abnormal cholesterol levels, which can contribute to dry eyes.

Prolonged Exposure to Video Display Terminals. Extended use of computers, smartphones, and other digital devices can reduce blink rate and tear production, leading to dry eyes.

Aging. Tear production tends to decrease with age, making older adults more susceptible to dry eyes.

Hormonal Changes. Hormonal changes, particularly in women during menopause, can affect tear production and lead to dry eyes.

Environmental Factors. Exposure to wind, smoke, dry air, and air conditioning can contribute to the evaporation of tears and result in dry eyes.

Blepharitis. Inflammation of the eyelids can affect the oil glands and lead to dry eyes.

Contact Lens Wear. Prolonged use of contact lenses can reduce tear production and cause dry eyes.

Autoimmune Diseases. Conditions such as rheumatoid arthritis and lupus can affect the lacrimal glands and lead to dry eyes.

Diabetes. High blood sugar levels can damage the nerves that control tear production, leading to dry eyes.

Vitamin A Deficiency. A lack of vitamin A can affect the health of the cornea and tear production, resulting in dry eyes.

Refractive Eye Surgeries: Procedures such as LASIK can temporarily or permanently affect tear production and lead to dry eyes.

Chronic Allergies, Allergic reactions can cause inflammation and reduce tear production, leading to dry eyes.

The disclosure also includes methods of treating inadequate saliva production and dry mouth. Medical conditions associated with inadequate saliva production and dry mouth include:

Sjögren's Syndrome. An autoimmune disorder characterized by dry eyes and dry mouth due to the body's immune system attacking its own moisture-producing glands.

Use of Drugs with Anticholinergic Effects. Medications such as antihistamines, antidepressants, and certain blood pressure medications can reduce saliva production and lead to dry mouth.

Radiotherapy for Head-and-Neck Tumors. Radiation treatment in the head and neck region can damage the salivary glands, leading to decreased saliva production and dry mouth.

Metabolic Syndrome. A cluster of conditions including high blood pressure, high blood sugar, excess body fat around the waist, and abnormal cholesterol levels, which can contribute to dry mouth.

Aging. Saliva production tends to decrease with age, making older adults more susceptible to dry mouth.

Hormonal Changes. Hormonal changes, particularly in women during menopause, can affect saliva production and lead to dry mouth.

Environmental Factors. Exposure to dry air and air conditioning can contribute to the evaporation of saliva and result in dry mouth.

Autoimmune Diseases. Conditions such as rheumatoid arthritis and lupus can affect the salivary glands and lead to dry mouth.

Diabetes. High blood sugar levels can damage the nerves that control saliva production, leading to dry mouth.

Vitamin A Deficiency. A lack of vitamin A can affect the health of the salivary glands and saliva production, resulting in dry mouth.

Refractive Eye Surgeries. Procedures such as LASIK can temporarily or permanently affect tear production.

Chronic Allergies. Allergic reactions can cause inflammation and reduce saliva production, leading to dry mouth.

Nerve Damage. Damage to the nerves that control the salivary glands, such as from surgery or injury, can lead to reduced saliva production and dry mouth.

Salivary Gland Infections. Infections of the salivary glands, such as mumps, can reduce saliva production and cause dry mouth.

The NMIIC Modulator can be administered by any pharmaceutically acceptable route. Ocular and oral administration are particularly contemplated.

The dosage range for eye drops or oral medication can vary depending on the specific medication, its concentration, the age or weight of the patient and the condition being treated. Below are some common dosage ranges for various types of eye drops. It is important to note that the specific dosage and frequency of eye drops should be determined by a healthcare professional based on the individual patient's condition and response to treatment. For ocular administration, 0.01 to 1000 mg of the NMIIC modulator may be administered daily. Administration can be 1, 2, 3 or 4 times daily, e.g. 1-2 drops per eye for each administration.

Oral drugs can be administered 1, 2, 3 or 4 times daily. Less frequent administration is preferred. Daily dosage ranges can be, e.g., 0.01 to 5000 mg of the NMIIC modulator, 1 to 1000 mg, 1 to 500 mg.

The therapeutically effective amount and frequency of administration of the non-muscle myosin IIC modulators disclosed herein may depend on various factors, including the medical condition being treated, the severity of the condition, the potency of the compound, the route of administration, the age, body weight, general health, gender and diet of the subject, and the response of the subject to the treatment, and can be determined by the treating physician.

In some embodiments, the non-muscle myosin IIC modulators of the pharmaceutical composition are present at a concentration of from about 100 nM to about 500 mM. Furthermore, the therapeutic agent can be present in an amount from about 1 nM to about 10 mM. Single doses of the pharmaceutical composition may contain from 0.1 qg to 0.1 g of the peptide, preferably 4 qg to 0.04 g or 400 qg to 0.4 g; and any effective amount of the therapeutic agent. Typically, single doses of the therapeutic agent range from 1 qg/kg body weight to 1000 mg/kg body weight (although lesser or greater dosages are also contemplated).

The dosing frequency may depend on, e.g., the route of administration chosen. For example, dosing by topical administration (e.g., by eye drop) may occur more frequently (e.g., 2, 3 or 4 times daily). To more quickly establish a therapeutic level of the non-muscle myosin IIC modulators, a loading dose of the non-muscle myosin IIC modulators that is greater (e.g., about 2- or 3-fold greater) than the maintenance dose can be administered at the beginning (e.g., in the first three days) of treatment followed by administration of the maintenance dose.

The non-muscle myosin IIC modulators can be administered via any suitable route, which may depend on, e.g., the medical condition being treated and its location and the pharmacokinetics of the inhibitor. Potential routes of administration of include without limitation oral, parenteral (including intradermal, subcutaneous, intramuscular, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]).

In some embodiments, the non-muscle myosin IIC modulators disclosed herein are administered orally. In other embodiments, non-muscle myosin IIC modulators are administered parenterally, such as intravenously, subcutaneously or intramuscularly. In further embodiments, non-muscle myosin IIC modulators are administered intratumorally to treat a tumor or cancer.

The length of treatment with non-muscle myosin IIC modulators can be determined by the treating physician to achieve the desired outcome. In some embodiments, non-muscle myosin IIC modulators are administered for at least about 1 week, 2 weeks, 3 weeks or 4 weeks (1 month). In other embodiments, non-muscle myosin IIC modulators are administered for at least about 6 weeks, 2 months, 3 months, 6 months, 1 year, 2 years, 3 years or longer.

### EXAMPLES

The following examples are intended only to illustrate the disclosure. Other synthetic processes, assays, studies, protocols, procedures, methodologies, techniques, reagents and conditions may alternatively be used as appropriate.

### Example 1: NMIIC Localizes to the Apical Junctions of Ductal Cells in Murine Lacrimal Gland

To determine the expression and localization of non-muscle myosin IIC (NMIIC) in the murine lacrimal gland, a series of experiments was conducted using NMIIC-GFP knockin mice and wildtype mice. The following methods and results were obtained.

RNA-seq Data Analysis: Previous RNA-seq data (accession number DRA010121 at DDBJ Sequenced Read Archive) was utilized to investigate the expression levels of myosin heavy chains in the murine lacrimal gland. The expression profile was calculated as transcripts per million (TPM).

Immunofluorescence Staining: Lacrimal glands were isolated from NMIIC-GFP mice and wildtype mice. The glands were fixed in 4% paraformaldehyde in phosphate-buffered saline (PBS; pH 7.4) and subjected to a sucrose gradient for cryoprotection. Cryosections (10-μm thick) were cut and adhered to glass slides. Sections were permeabilized, blocked, and incubated with primary antibodies against NMIIA, AQP5, or ZO-1, followed by secondary antibodies conjugated with Alexa Fluor dyes. Phalloidin was used to label F-actin, and DAPI was used to label nuclei. Microscopy was performed using a Nikon ECLIPSE Ti2 inverted fluorescence microscope.

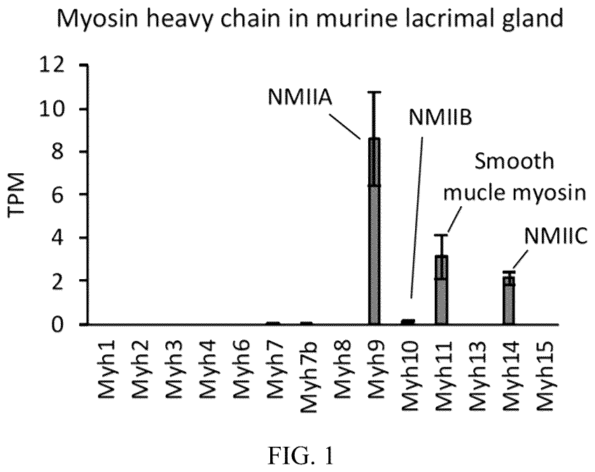

Expression of Myosin Heavy Chains: RNA-seq data revealed that the predominant myosin heavy chains expressed in the murine lacrimal gland are Myh9 (NMIIA) and Myhl4 (NMIIC), while Myh10 (NMIIB) expression was substantially lower. Myh11, a smooth muscle myosin-heavy chain, was also expressed, suggesting its presence in myoepithelial cells (FIG. 1).

Localization of NMIIA: Immunofluorescence staining of NMIIA in wildtype mice showed that NMIIA was expressed in acinar cells, where it localized to secretory granules fused to the apical membrane during carbachol-induced exocytosis. This localization pattern is similar to that observed in the salivary gland, indicating a role for NMIIA in extruding granule contents across exocrine glands.

Localization of NMIIC: In NMIIC-GFP mice, NMIIC-GFP was enriched at the apical junctions of ductal cells and expressed at much lower levels in acinar cells. High magnification images revealed that NMIIC at the ductal cell apical junctions was expressed as regularly-spaced puncta along F-actin. Detailed analysis of fluorescence intensity across the apical junction of ductal cells indicated that fluorescence intensity peaks of F-actin and NMIIC flanked and partially overlapped with that of ZO-1, suggesting some level of interaction between these proteins.

These results demonstrate that NMIIC is predominantly localized at the apical junctions of ductal cells in the murine lacrimal gland, where it colocalizes with tight junction proteins such as ZO-1. This localization pattern supports the hypothesis that NMIIC plays a role in regulating tight junction integrity and, consequently, tear secretion through a paracellular pathway.

### Example 2: Carbachol-Induced Tear Volume is Increased in NMIIC-KO Mice Via Tight Junction Disruption

To investigate the role of non-muscle myosin IIC (NMIIC) in tear secretion, tear volume in NMIIC knockout (NMIIC-KO) mice and wild-type (WT) mice was measured by stimulating surgically exposed lacrimal glands with carbachol. The following methods and results were obtained.

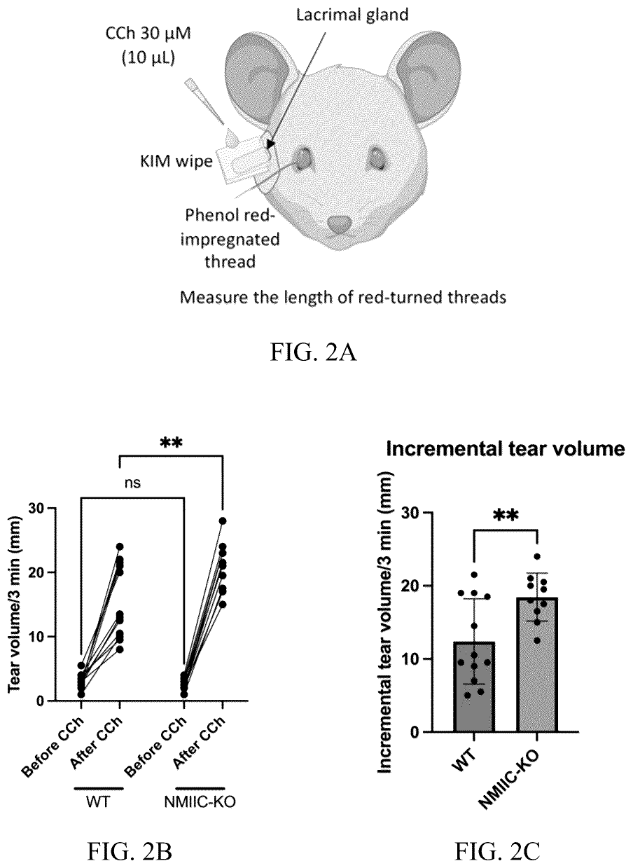

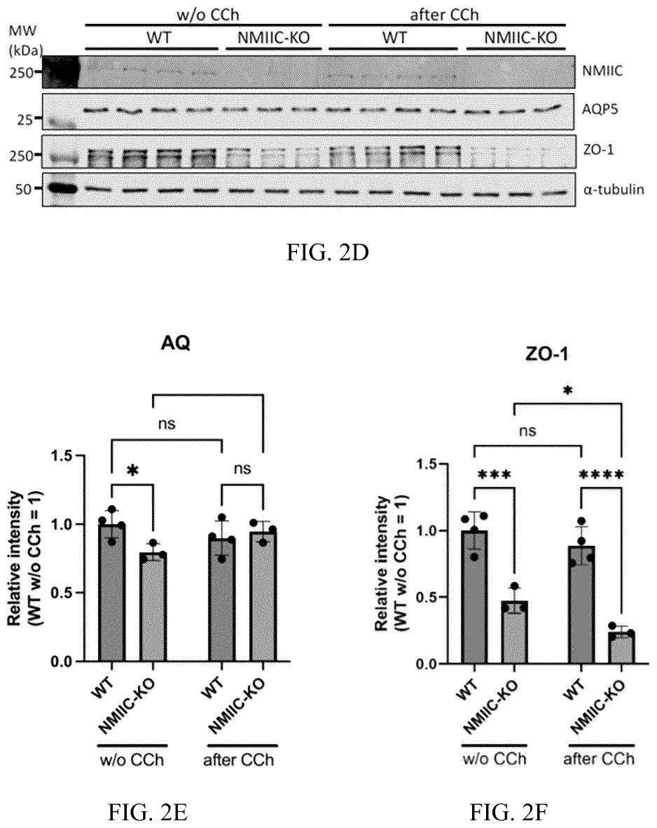

Measurement of Tear Volume: As shown in FIG. 2A, the tear volume was determined using the cotton thread test with phenol red-impregnated threads. Mice were anesthetized by intraperitoneal (i.p.) injection of an anesthetic agent mixture (0.75 mg/kg of medetomidine, 4.0 mg/kg of midazolam, and 5.0 mg/kg of butorphanol) at a volume of 0.05 mL/10 g body weight. The mice were placed on a heating pad, and lacrimal glands were exposed under anesthesia and wrapped with a small cut of cellulose mesh. Carbachol solution was applied topically on the lacrimal glands at a concentration of 0.3 M and a volume of 10 μL. The volume of tear fluid was measured by carefully placing a phenol red-impregnated thread at the canthus of each eye for 3 minutes before and after carbachol stimulation. The length of the thread that changed color due to absorption of tear fluid was measured in millimeters. The incremental tear volume was calculated by subtracting the tear volume before carbachol stimulation from the tear volume after carbachol stimulation.

Immunofluorescence Staining: Lacrimal glands were isolated from NMIIC-KO mice and WT mice. The glands were fixed in 4% paraformaldehyde in phosphate-buffered saline (PBS; pH 7.4) and subjected to a sucrose gradient for cryoprotection. Cryosections (10-μm thick) were cut and adhered to glass slides. Sections were permeabilized, blocked, and incubated with primary antibodies against AQP5 or ZO-1, followed by secondary antibodies conjugated with Alexa Fluor dyes. Phalloidin was used to label F-actin, and DAPI was used to label nuclei. Microscopy was performed using a Nikon ECLIPSE Ti2 inverted fluorescence microscope.

Western Blot Analysis: Lacrimal glands were homogenized in ice-cold RIPA lysis buffer containing protease inhibitors. The homogenates were incubated on ice for 15 minutes and then spun at 14,000 g for 10 minutes. Supernatants were collected, and protein concentrations were determined by the Bradford method. Twenty-μg protein samples were separated by SDS-PAGE and transferred onto a nitrocellulose membrane. The blots were blocked in 5% skim milk and probed with primary antibodies against NMIIC, AQP5, ZO-1, or α-tubulin, followed by secondary antibodies conjugated with Alexa Fluor dyes. Images were acquired using an Odyssey CLx Imager, and band intensities were measured with Image Studio software.

Tear Volume Measurement: Basal tear volume before carbachol stimulation did not show a significant difference between NMIIC-KO mice and WT mice (FIG. 2B; WT vs. NMIIC-KO=3.1±1.1 vs. 2.5±0.9 mm/3 min). However, NMIIC-KO mice showed significantly increased tear secretion after carbachol stimulation compared to WT mice (FIG. 2B; WT vs. NMIIC-KO=15.5±5.8 vs. 21.0±3.8 mm/3 min). The incremental tear volume also showed a significant increase in the NMIIC-KO group (FIG. 2C; WT vs. NMIIC-KO=12.4±5.8 vs. 18.5±3.3 mm/3 min).

Localization of AQP5 and ZO-1: Immunofluorescence staining showed that AQP5 was mainly expressed on the apical membrane of the duct and at lower levels on the apical membrane of acinar cells in the lacrimal gland of WT mice. This expression pattern was not affected in NMIIC-KO mice. ZO-1 was expressed at the apical junctions of both ductal cells and acinar cells. NMIIC loss did not affect the localization of ZO-1.

Protein Levels of AQP5 and ZO-1: Western blot analysis confirmed the knockout of NMIIC in NMIIC-KO mice. AQP5 protein levels showed only a slight decrease in the NMIIC-KO lacrimal gland without carbachol stimulation and remained unchanged after carbachol stimulation (FIG. 2D and FIG. 2E; WT vs. NMIIC-KO=1.00±0.10 vs. 0.80±0.06 without carbachol stimulation, and 0.90±0.13 vs. 0.95±0.07 after carbachol stimulation). Conversely, ZO-1 levels were notably reduced by 53% and 73% in the NMIIC-KO lacrimal gland compared to WT, without and after carbachol stimulation, respectively (FIG. 2D and FIG. 2F; WT vs. NMIIC-KO=1.00±0.14 vs. 0.47±0.10 without carbachol stimulation, and 0.89±0.14 vs. 0.24±0.04 after carbachol stimulation).

These data suggest that the mechanism underlying the increase in carbachol-induced tear volume in mice lacking NMIIC involves reduced ZO-1 expression, leading to tight junction disruption. This supports the hypothesis that NMIIC regulates tear secretion through modulation of tight junction permeability.

### Example 3: Tears from NMIIC-KO Mice Showed Brighter Fluorescence when a Fluorescent Dye was Mixed with Carbachol

To further investigate the role of non-muscle myosin IIC (NMIIC) in tear secretion and the potential for paracellular water leakage, an experiment was conducted where a fluorescent dye was mixed with carbachol to stimulate tear secretion in NMIIC knockout (NMIIC-KO) mice and wild-type (WT) mice. The following methods and results were obtained:

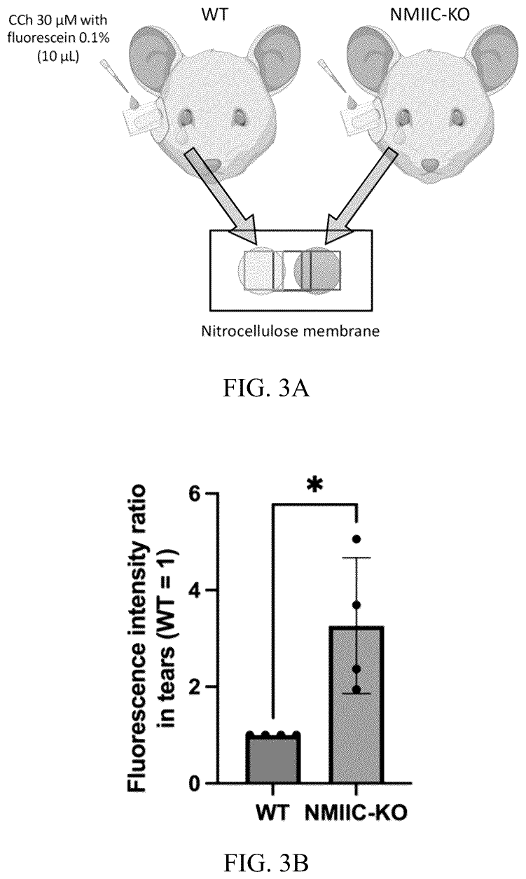

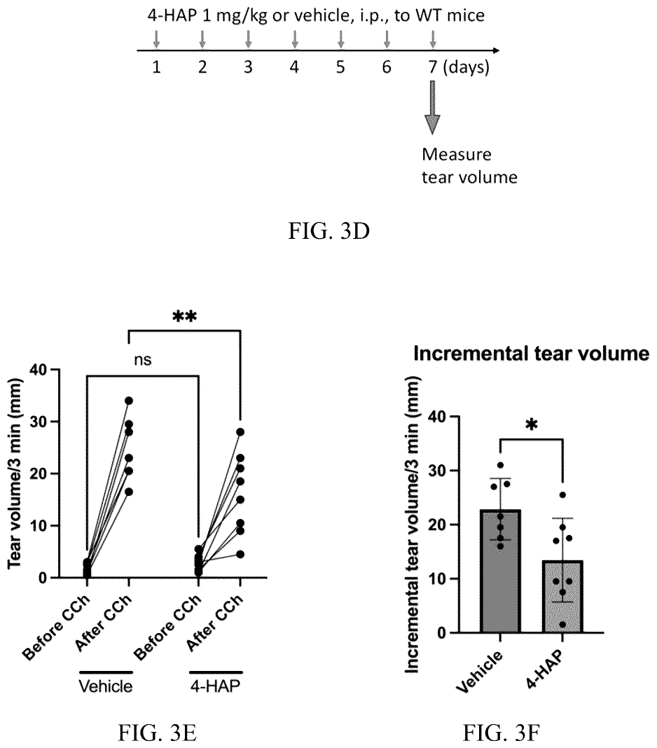

Fluorescent Dye Experiment: As shown in FIG. 3A, mice were anesthetized by intraperitoneal (i.p.) injection of an anesthetic agent mixture (0.75 mg/kg of medetomidine, 4.0 mg/kg of midazolam, and 5.0 mg/kg of butorphanol) at a volume of 0.05 mL/10 g body weight. The mice were placed on a heating pad, and lacrimal glands were exposed under anesthesia and wrapped with a small cut of cellulose mesh. Carbachol solution mixed with fluorescein (a fluorescent compound with a small molecular weight, which is hydrophilic and thus impermeable to cell membranes) was applied topically on the lacrimal glands at a concentration of 0.3 M carbachol and 0.1% fluorescein, and a volume of 10 μL. Tears were collected simultaneously from NMIIC-KO and WT mice on a nitrocellulose membrane and observed under a microscope.

Fluorescence Intensity Measurement: The collected tears on the nitrocellulose membrane were observed under a microscope to assess fluorescence intensity. Images of the tears were captured, and the relative fluorescence intensity was quantified using image analysis software.

Fluorescence Intensity in Tears: Tears from NMIIC-KO mice appeared more strongly fluorescent compared to those from WT mice. The quantification of fluorescence intensity from the tears confirmed a significant increase in fluorescence in the NMIIC-KO group compared to the WT group (FIG. 3B; WT vs. NMIIC-KO=1.00±0.00 vs. 3.26±1.41). This indicates that the tears from NMIIC-KO mice contained higher levels of fluorescein, suggesting increased paracellular leakage.

These data provide evidence for increased paracellular water leakage in the lacrimal glands of NMIIC-KO mice. The higher fluorescence intensity in the tears of NMIIC-KO mice, when stimulated with carbachol mixed with fluorescein, supports the hypothesis that NMIIC regulates tight junction integrity and that its absence leads to increased permeability and tear secretion through a paracellular pathway.

### Example 4. Activation of NMIIC by 4-Hap Reduces Carbachol-Induced Tear Volume

To further confirm the role of non-muscle myosin IIC (NMIIC) in regulating tear secretion, the effect of pharmacological activation of NMIIC was investigated using 4-Hydroxyacetophenone (4-HAP) on carbachol-induced tear volume in wild-type (WT) mice. The following methods and results were obtained:

4-HAP Treatment Protocol: 4-HAP was dissolved in ethanol and then diluted 100-fold with phosphate-buffered saline (PBS). WT mice were administered 4-HAP intraperitoneally (i.p.) at a dosage of 1 mg/kg (volume: 0.05 mL/10 g body weight) once a day for 7 consecutive days (as shown in FIG. 3D). The vehicle group was administered the same volume of PBS containing 1.0% ethanol. Tear volume measurement and isolation of the lacrimal glands were performed on the last day of injection.

Measurement of Tear Volume: The tear volume was determined using the cotton thread test with phenol red-impregnated threads. Mice were anesthetized by intraperitoneal (i.p.) injection of an anesthetic agent mixture (0.75 mg/kg of medetomidine, 4.0 mg/kg of midazolam, and 5.0 mg/kg of butorphanol) at a volume of 0.05 mL/10 g body weight. The mice were placed on a heating pad, and lacrimal glands were exposed under anesthesia and wrapped with a small cut of cellulose mesh. Carbachol solution was applied topically on the lacrimal glands at a concentration of 0.3 μM and a volume of 10 μL. The volume of tear fluid was measured by carefully placing a phenol red-impregnated thread at the canthus of each eye for 3 minutes before and after carbachol stimulation. The length of the thread that changed color due to absorption of tear fluid was measured in millimeters. The incremental tear volume was calculated by subtracting the tear volume before carbachol stimulation from the tear volume after carbachol stimulation.

Effect of 4-HAP on Tear Volume: 4-HAP-treated mice showed reduced tear volume after carbachol stimulation compared to vehicle-treated mice (FIG. 3E; WT vs. 4-HAP=24.6±6.1 vs. 16.2±7.9 mm/3 min), while there was no significant difference between these two groups before carbachol stimulation (FIG. 3E; WT vs. 4-HAP=1.7±1.1 vs. 2.8±1.6 mm/3 min). The incremental tear volume in 4-HAP-treated mice also showed a significant decrease compared to vehicle-treated mice (FIG. 3F; WT vs. 4-HAP=22.9±5.7 vs. 13.4±7.7 mm/3 min).

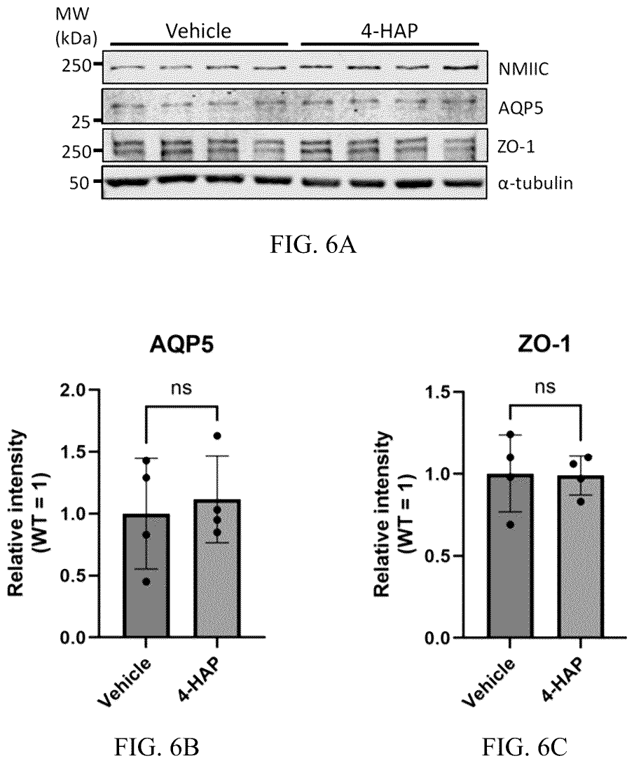

Localization and Expression of AQP5 and ZO-1: To determine whether the localization or expression levels of AQP5 and ZO-1 were affected by 4-HAP treatment, immunofluorescence staining and western blot analysis were performed. NMIIC, AQP5, and ZO-1 localization did not change between the 4-HAP and vehicle groups. Western blot analysis showed that 4-HAP treatment did not change the expression levels of AQP5 and ZO-1 (FIG. 6A, FIG. 6B, and FIG. 6C).

These results demonstrate that pharmacological activation of NMIIC by 4-HAP reduces carbachol-induced tear secretion in WT mice. The reduction in tear volume without changes in the localization or expression levels of AQP5 and ZO-1 suggests that 4-HAP-mediated activation of NMIIC enhances tight junction integrity, thereby reducing paracellular water permeability and tear secretion. This supports the hypothesis that NMIIC plays a critical role in regulating tear secretion through modulation of tight junction permeability.

### Example 5. NMIIC Loss Reduces Water Secretion in Salivary Gland

To gain further insight into NMIIC-mediated regulation of tight junctions in exocrine glands, the role of NMIIC in saliva secretion in the parotid gland was investigated and compared with the lacrimal gland data. The following methods and results were obtained:

Expression Level of NMIIC: The expression level of NMIIC in the parotid gland was compared to that in the lacrimal gland using western blot analysis.

Immunofluorescence Staining: Parotid glands were isolated from NMIIC-GFP mice and wild-type (WT) mice. The glands were fixed in 4% paraformaldehyde in phosphate-buffered saline (PBS; pH 7.4) and subjected to a sucrose gradient for cryoprotection. Cryosections (10-μm thick) were cut and adhered to glass slides. Sections were permeabilized, blocked, and incubated with primary antibodies against AQP5 or ZO-1, followed by secondary antibodies conjugated with Alexa Fluor dyes. Phalloidin was used to label F-actin, and DAPI was used to label nuclei. Microscopy was performed using a Nikon ECLIPSE Ti2 inverted fluorescence microscope.

Measurement of Saliva Volume: The saliva volume was determined using a gravimetric method with paper plugs. Mice were anesthetized by intraperitoneal (i.p.) injection of an anesthetic agent mixture (0.75 mg/kg of medetomidine, 4.0 mg/kg of midazolam, and 5.0 mg/kg of butorphanol) at a volume of 0.05 mL/10 g body weight. The mice were placed on a heating pad, and parotid glands were exposed under anesthesia and wrapped with a small cut of cellulose mesh. Carbachol solution was applied topically on the parotid glands at a concentration of 0.3 M and a volume of 20 μL. The secreted saliva was absorbed into paper plugs inserted into the oral cavity and exchanged at 3-minute intervals up to 15 minutes after starting carbachol stimulation. The saliva-saturated plugs were weighed and corrected for the original weight of the paper plug. The volume of secreted saliva was calculated as the increase in weight of each paper plug. The total saliva volume was calculated by summing up the increase in weight of each paper plug after carbachol stimulation.

Western Blot Analysis: Parotid glands were homogenized in ice-cold RIPA lysis buffer containing protease inhibitors. The homogenates were incubated on ice for 15 minutes and then spun at 14,000 g for 10 minutes. Supernatants were collected, and protein concentrations were determined by the Bradford method. Twenty-μg protein samples were separated by SDS-PAGE and transferred onto a nitrocellulose membrane. The blots were blocked in 5% skim milk and probed with primary antibodies against NMIIC, AQP5, ZO-1, or α-tubulin, followed by secondary antibodies conjugated with Alexa Fluor dyes. Images were acquired using an Odyssey CLx Imager, and band intensities were measured with Image Studio software.

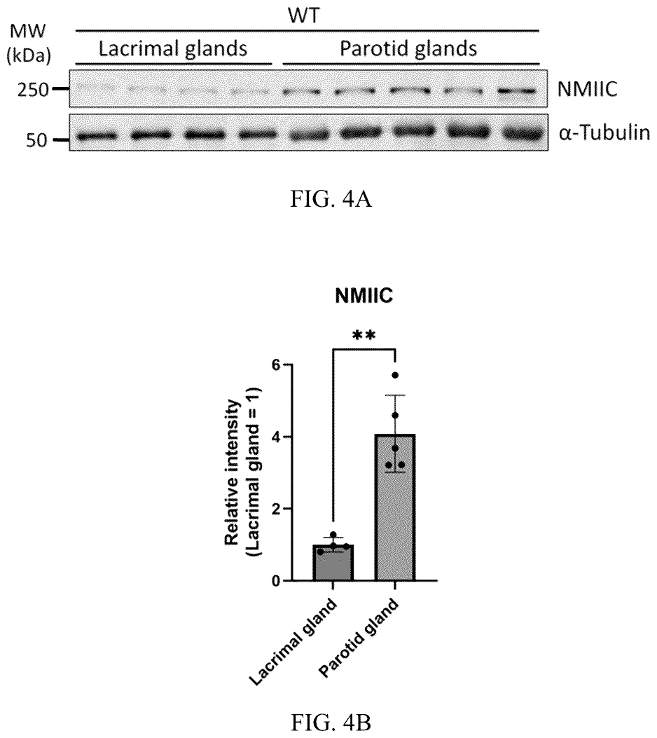

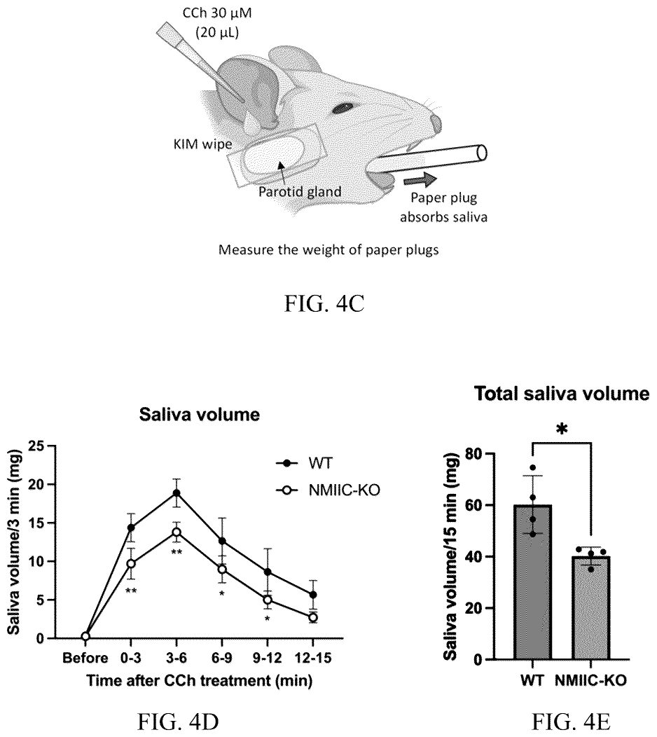

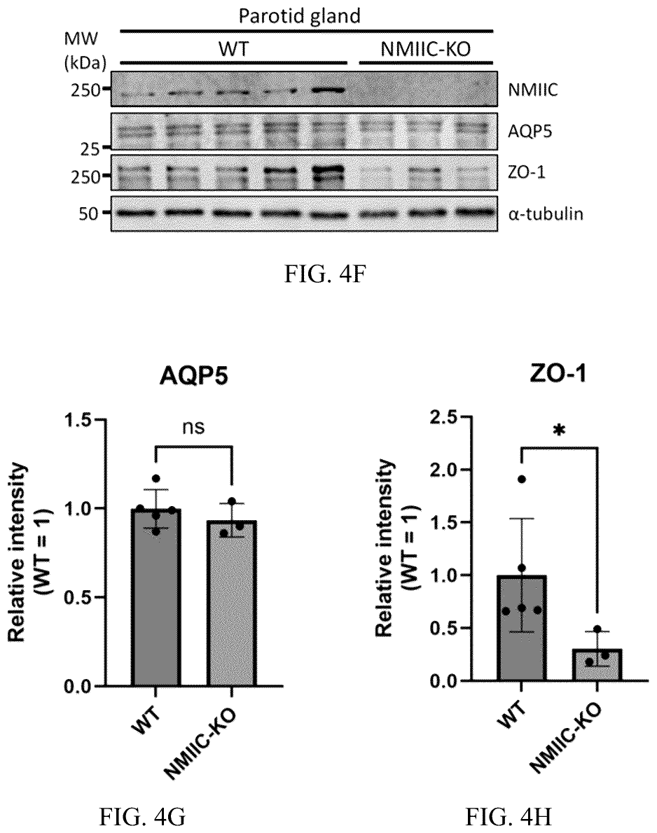

Expression Level of NMIIC: The expression level of NMIIC in the parotid gland was higher than that in the lacrimal gland (FIG. 4A and FIG. 4B; lacrimal gland vs. parotid gland =1.00±0.20 vs. 4.09±1.07).

Localization of NMIIC: In the parotid gland of NMIIC-GFP mice, NMIIC-GFP fluorescence was enriched at the apical junction of ductal cells and was less expressed in acinar cells. The localization and pattern of NMIIC, in puncta, was similar to that of NMIIC in the lacrimal gland.

Saliva Secretion: NMIIC-KO mice showed decreased saliva secretion after carbachol stimulation compared to WT mice (FIG. 4C, FIG. 4D and FIG. 4E; WT vs. NMIIC-KO=60.2±11.3 vs. 40.2±3.5 mg/15 min).

Localization of AQP5 and ZO-1: Immunofluorescence staining showed that AQP5 was mainly expressed on the apical membrane of the acinar cells and not expressed at apical junctions of ductal cells in the parotid gland of WT mice. This localization was not affected in NMIIC-KO mice. ZO-1 was enriched at the apical junctions between ductal cells and at a lower level between acinar cells. NMIIC-KO did not affect the localization of ZO-1.

Protein Levels of AQP5 and ZO-1: Western blot analysis showed that ZO-1 was reduced by 70% in the NMIIC-KO parotid gland compared to WT (FIG. 4F and FIG. 4H; WT vs. NMIIC-KO=1.00±0.54 vs. 0.30±0.16), whereas AQP5 remained unchanged (FIG. 4F and FIG. 4G; WT vs. NMIIC-KO=1.00±0.11 vs. 0.93±0.10).

While NMIIC loss resulted in a significant reduction in ZO-1 expression in both lacrimal and parotid glands, the opposing consequences of increased tear volume but reduced saliva volume likely reflect distinct mechanisms of secretion and degrees of paracellular water permeability between these organs. The data revealed that paracellular water secretion occurs in acinar cells but not in ducts in the healthy salivary gland.

### DISCUSSION AND SIGNIFICANCE

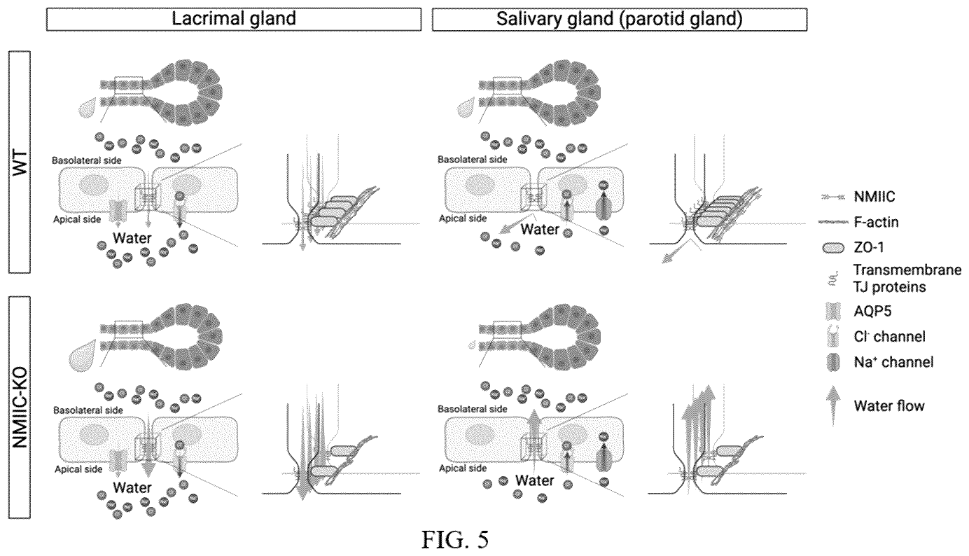

Disclosed herein is a paracellular water secretion pathway in tear secretion by targeting NMIIC, which was expressed at apical junctions of ductal cells in the murine lacrimal gland, overlapping with tight junctions. It was found that tear volume after carbachol stimulation was significantly increased in NMIIC-KO mice. Moreover, when mixing a fluorescent dye with carbachol, tears from NMIIC-KO mice showed brighter fluorescence. Administration of an NMIIC activator, 4-HAP, to wildtype mice, inhibited tear secretion. The localization of AQP5 and the tight junction protein ZO-1 in lacrimal glands by immunofluorescence were unchanged in NMIIC-KO mice. However, ZO-1 protein levels were significantly reduced in NMIIC-KO lacrimal glands, while AQP5 levels remained unchanged. Intriguingly, contrary to tear secretion, carbachol-induced saliva secretion was reduced in NMIIC-KO mice, even with reduced ZO-1 expression. In summary, disclosed findings reveal a new mechanism for paracellular water secretion in the lacrimal gland via NMIIC-mediated regulation of ductal cell tight junctions, which seems not to exist in the parotid gland (as illustrated schematically in FIG. 5).

Water Secretion in the Lacrimal Gland Duct. The ducts in the lacrimal gland are reported to secrete water. In the murine lacrimal gland, the dominant water channel for transcellular water secretion is thought to be AQP5 based on previous reports as well as previous RNA-seq data (accession number DRA010121 at DDBJ Sequenced Read Archive). Recently, single-cell RNA-seq data revealed that AQP5 is more highly expressed in ductal cells compared to acinar cells in the murine lacrimal gland. This is consistent with data showing that ductal cells have higher AQP5 expression in their apical membrane than acinar cells, and also consistent with previous papers in which AQP5 was strongly stained in ducts compared to acini. The single-cell RNA-seq data also suggested that ductal cells have several ion channels and ion transporters as well as Na+-K+ ATPase, which have lower, or no expression in acinar cells. This also suggests that the lacrimal ducts are responsible for water secretion since the secreted ions create higher osmotic pressure in the ductal lumen side than in extracellular fluid or cytosol in ductal cells and make water move into the lumen side of the duct. Therefore, the possibility of paracellular water secretion in the “duct” is also reasonable. Acinar cells in the lacrimal gland appear to mediate protein secretion via exocytosis when muscarinic receptors are stimulated by carbachol, although it remains unclear whether these cells also contribute to water secretion.

Possibility of Paracellular Water Secretion from Between Ductal Cells in the Lacrimal Gland. NMIIC-KO mice showed increased tear secretion and 4-HAP-injected mice showed decreased tear secretion. Moreover, after stimulating the lacrimal gland with a fluorescein-containing carbachol solution, tears from NMIIC-KO mice were significantly more fluorescent than wild-type. Since fluorescein, a fluorescence compound with a small molecular weight, is hydrophilic and thus impermeable to cell membranes, these data suggest either: 1) paracellular water secretion is upregulated/downregulated depending on NMIIC status, or 2) the endocytosis of extracellular fluid from basolateral side and exocytosis of the granules into the lumen side are upregulated/downregulated depending on NMIIC status. However, since ZO-1 expression levels are reduced in NMIIC-KO mice and the expression levels of NMIIC in the acinar cells, where much higher exocytosis activity exists, is significantly lower than in ductal cells, the first hypothesis is more plausible.

Different Paracellular Water Permeability of Duct in Lacrimal Gland and Salivary Gland. There is currently no study that reports evidence in support of the existence of the paracellular water pathway in tear secretion. On the other hand, there are some reports regarding this pathway in the salivary gland. Murakami et al. reported the possibility of paracellular water transport based on a mathematical model fitting to the experimental permeability data and exposure to a hypertonic solution in perfused rat submandibular glands. Experiments analyzing the perfusion of lucifer yellow, a cellular impermeable fluorescence substance, with some saliva stimulants, also support the presence of a paracellular water pathway in saliva secretion. Therefore, it is not unlikely that paracellular water secretion occurs in the lacrimal gland. However, a major distinction highlighted by study is that paracellular water secretion in the salivary gland is considered to occur between acinar cells, because saliva secreted into the oral cavity is known to be hypotonic after reabsorption of ions from primary saliva in the duct, and thus the junction of duct should be sealed tightly. If paracellular water permeability were high in the duct in the salivary gland, then saliva would become close to isotonic fluid during the flow of primary fluid from acini towards the duct. It was hypothesized that this to be the mechanism underlying reduced saliva secretion in absence of NMIIC, specifically that NMIIC-KO loosened the tight junction via reduced ZO-1 expression level causing water to be reabsorbed through the ductal junctions due to osmotic pressure differences between hypotonic primary saliva and isotonic extracellular fluid. The data thus revealed that paracellular water secretion occurs in acinar cells but not in ducts in the healthy salivary gland. On the other hand, tears are reported to be isotonic fluid, supporting the likelihood of paracellular water transport in the duct. These differences are also supported by the opposite localization of ion/water channels such as chloride channels and AQP5 as well as ion transporters such as NKCC1; they are enriched in ductal cells in the lacrimal gland but in acinar cells in the salivary gland. Therefore, the paracellular water permeability in ducts is considered to be high in the lacrimal gland but quite low in the salivary gland.

Mechanism of NMIIC-Mediated Regulation of Paracellular Water Secretion in the Lacrimal Gland. 4-HAP is known to activate NMIIB and NMIIC, but it is considered to be exclusively effective on NMIIC in this study because NMIIC mRNA is expressed in the murine lacrimal gland but NMIIB mRNA expression levels are negligible. Activation by 4-HAP causes an increase in NMIIC assembly in cells. Based on this fact, two possibilities were proposed: one is that 4-HAP-treatments results in increased NMIIC-forces on the actomyosin belt that overlaps with the tight junction, thus increasing barrier integrity and reducing tear secretion. The other possibility is that activating NMIIC enables the recruitment of tight junction proteins, thus influencing tear volume depending on the tight junction expression level. The results revealed that the former is more likely as there was no significant difference in ZO-1 expression levels after 4-HAP treatment. However, the latter was could be possible if 4-HAP was administered to mice for a longer duration because several studies report that changing forces on tight junctions can alter their composition and function; e.g., physiological shear stress was reported to increase the expression level of tight junctions in brain microvascular endothelial cells, and junctional tension was also reported to lead to the transportation of non-junctional ZO-1 toward tight junctions in zebrafish embryos. Furthermore, myosin light chain is reported to be related to forming tight junctions. These reports provide mechanistic insight into NMIIC-regulating tightening of tight junctions which leads to changing volume of paracellular water transport, although further study is needed to confirm this.

Translational Perspective. Dry eye is a condition that continues to increase in prevalence, especially as tear volume in humans tends to decrease with age. Identification of NMIIC as a direct modulator of water secretion through a paracellular pathway in the ductal cells of the lacrimal and salivary glands establishes a promising target for the development of pharmaceutical therapeutics for dry eye or dry mouth.

Disclosed findings are the first to provide evidence for the involvement of a paracellular water pathway in tear secretion. Furthermore, it was found that this pathway is regulated by NMIIC via modulation of tight junctions in the ductal cells in the lacrimal glands and salivary glands. This study also highlights NMIIC as a potential target for the development of treatments for dry eye/mouth syndrome.

What is claimed is:

1. A method of treating a condition associated with tear production in a subject, comprising administering to the subject a therapeutically effective amount of a non-muscle myosin IIC (NMIIC) modulator, wherein the NMIIC modulator is an NMIIC inhibitor.

2. The method of claim 1, wherein the NMIIC inhibitor comprises blebbistatin (CAS Reg. No. 856925-71-8), MT-228 (CAS Reg. No. 2404652-24-8), or a pharmaceutically acceptable salt, solvate, or hydrate thereof of any of the foregoing.

3. The method of claim 1, wherein the condition comprises a dry eye syndrome, or a Sjögren's syndrome.

4. The method of claim 1, wherein the NMIIC modulator is administered topically, orally, parenterally, subcutaneously, intradermally, intramuscularly, or by implantation.

5. The method of claim 1, wherein the NMIIC inhibitor is administered topically to an ocular surface of the subject.

6. The method of claim 1, wherein the NMIIC modulator is in a form of an ophthalmic solution, a suspension, a gel, an ointment, or an emulsion.

7. The method of claim 1, wherein the NMIIC inhibitor is administered via a unit-dose sterile ampoule, a multi-dose preservative-free eye drop container, or an ophthalmic squeeze dispenser.

8. The method of claim 1, wherein administering the NMIIC inhibitor comprises once-daily, twice-daily, or thrice-daily dosing.

9. The method of claim 1, wherein the NMIIC inhibitor is administered at a concentration from 0.0001% to 5% weight/volume (w/v).

10. The method of claim 1, wherein the therapeutically effective amount comprises a dose of 0.001 mg/kg to 50 mg/kg of the NMIIC inhibitor.

11. The method of claim 1, wherein the therapeutically effective amount is defined as an amount sufficient to increase tear volume, increase aqueous tear secretion, or improve tear film stability relative to a pre-treatment baseline.

12. The method of claim 1, wherein administering the NMIIC inhibitor comprises delivering a controlled-release topical ocular dosage form that comprises a gel-forming in situ system, an ocular insert, a hydrogel depot, or a biodegradable carrier.

13. The method of claim 1, wherein the NMIIC inhibitor is co-administered with an additional therapeutic agent selected from a lubricant, a corticosteroid, an immunomodulator, or an anti-inflammatory agent.

14. A method of increasing tear production in a subject, comprising administering to the subject a therapeutically effective amount of a non-muscle myosin IIC (NMIIC) modulator, wherein the NMIIC modulator is an NMIIC inhibitor.

15. The method of claim 14, wherein the NMIIC inhibitor comprises Blebbistatin (CAS Reg. No. 856925-71-8), MT-228 (CAS Reg. No. 2404652-24-8), or a pharmaceutically acceptable salt of any of the foregoing.

16. The method of claim 14, wherein the NMIIC modulator is administered topically, orally, parenterally, subcutaneously, intradermally, intramuscularly, or by implantation.

17. A method of treating a condition associated with saliva production in a subject, comprising administering to the subject a therapeutically effective amount of a non-muscle myosin IIC modulator, wherein the NMIIC modulator is an NMIIC activator.

18. The method of claim 17, wherein the NMIIC activator comprises 4-hydroxyacetophenone (CAS Reg. No. 99-93-4), or a pharmaceutically acceptable salt, solvate, or hydrate thereof.

19. The method of claim 8, wherein the NMIIC modulator is administered topically, orally, parenterally, subcutaneously, intradermally, intramuscularly, by implantation, by intracavitary, or intradermally.

20. The method of claim 17, wherein the NMIIC activator in a form of a mucosal gel, an oral rinse, a spray, a film, a paste, or a bioadhesive formulation.
